(19) United States
(12) Patent Application Publication
(10) Pub. No.: US 2025/0185949 A1
(43) Pub. Date: Jun. 12, 2025

(54) REAL-TIME NON-INVASIVE BLOOD GLUCOSE MONITORING DEVICE WITH ENHANCED VSWR-BASED MEASUREMENT

(71) Applicants: Pallob Kumar Gain, Khulna (BD); Amilkar Nunez Rosales, Houston, TX (US)

(72) Inventors: Pallob Kumar Gain, Khulna (BD); Amilkar Nunez Rosales, Houston, TX (US)

(21) Appl. No.: 18/530,696

(22) Filed: Dec. 6, 2023

(51) Int. Cl. A61B 5/145; A61B 5/00; A61B 5/05

(52) U.S. Cl. CPC A61B 5/14532; A61B 5/0015; A61B 5/05; A61B 5/7275; A61B 2560/0223; A61B 2560/0443

(57) ABSTRACT

The present invention discloses a state-of-the-art non-invasive blood glucose monitoring system powered by radio frequency (RF) signals to determine glucose levels in the blood. The system consists of a sensor plate, which functions as an antenna, transmitting RF signals through the body and analyzing the reflection coefficient to detect changes in blood glucose concentration. A Voltage Standing Wave Ratio (VSWR) meter is integrated with the device to measure the reflection of the RF signal, which is indicative of the body's impedance mismatch due to glucose variations. The system incorporates a microcontroller, which processes the forward and reverse voltage signals obtained from the VSWR meter to calculate the reflection coefficient. In addition, a Bluetooth system is included for wireless data transmission to a secondary device, such as a smartphone or PC, where a specialized software application processes the VSWR data to estimate the blood glucose level through quadratic interpolation. The efficacy of the system is validated against standard glucometer readings, demonstrating its potential as a convenient and pain-free alternative for diabetes management and monitoring. The device's architecture and its components are designed to ensure user-friendly operation, precise measurements, and seamless integration into the user's lifestyle.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The present invention pertains to the field of medical devices, specifically a non-invasive glucometer system that measures Blood Glucose Levels (BGL) without necessitating the extraction of a blood sample. The device is designed to achieve superior accuracy and performance through the use of High-Frequency (HF) radio waves. The radio wave impedance of human tissues is intrinsically linked to the plasma layer electric permittivity of cells, which, in turn, is influenced by the level of BGL. This innovative technology represents a significant advancement in the field of medical devices, offering a non-invasive and highly accurate means of monitoring BGL levels.

### Description of the Related Art

The prevalence of diabetes mellitus, a multifaceted metabolic disorder, has seen a dramatic global upsurge, intensifying the need for efficient management strategies. World Health Organization reports indicate a surge from 108 million individuals in 1980 to 422 million in 2014, with projections suggesting an escalation to 629 million by 2045. Notably, the incidence rate among adults over 18 years has doubled, climbing from 4.7% in 1980 to 8.5% in 2014. Diabetes arises either due to inadequate insulin production by the pancreas or the body's ineffective use of insulin, leading to hyperglycemia. Chronic hyperglycemia precipitates severe systemic damage over time, notably to cardiovascular and nervous systems, culminating in an enhanced likelihood of premature mortality. Complications are diverse and profound, including heightened risks of renal failure, cardiovascular incidents, retinal damage, neuropathy, and extremity amputations. Additionally, diabetes imposes serious risks during pregnancy, such as fetal morbidity and associated obstetric complications. With its three primary classifications, diabetes remains a significant global health challenge necessitating innovations in its detection and control.

Conventional methodologies for Blood Glucose Level (BGL) measurement are predominantly invasive, often requiring clinical settings for accurate assessments. The glucometer, a prevalent tool, necessitates a blood sample, typically procured from a finger prick. This sample is then placed onto a test strip and analyzed by the glucometer to yield glucose concentration values. Such invasive methods are not without their drawbacks, including discomfort, pain, the risk of infection, and the financial burden of single-use test strips. Moreover, the psychological aversion to blood and needles for some individuals cannot be understated.

Moreover, by looking at prior art, multiple advancements, techniques and devices have been seen. For instance, a U.S. Pat. No. 2,008,0097170A1 relates to Blood glucose monitoring system. The blood glucose monitoring system, comprising (i) a blood glucose monitor for monitoring a blood glucose level and for producing digitally encoded blood glucose level signals representative of the blood glucose level, (ii) a programmable microprocessor-based portable unit, (iii) digital data storage media tangibly embodying therein processor-executable program instructions to signal process in response to signals based upon the digitally encoded blood glucose level signals and further to signal process insulin dosage data, and calibration information, (iv) a signal interface connected in signal communication with the programmable microprocessor-based portable unit and the blood glucose monitor for directly coupling the digitally encoded blood glucose level signals supplied by the blood glucose monitor to the programmable microprocessor-based portable unit, and (v) a signal processor for performing signal processing functions in accordance with the program instructions.

A U.S. Pat. No. 5,019,974A relates to diabetes management systems and apparatus. The system and apparatus for efficient medical control of a medical condition such as diabetes comprises a recorder, an interface and a master computer. The master computer develops a program of therapy which is downloaded into the recorder which then reminds the patient of any therapy due and records that the therapy has been effected. The record from the recorder is subsequently fed back to the master computer to improve or alter the therapy program.

A U.S. Pat. No. 7,976,778B2 relates to blood glucose tracking apparatus. The measurement module for glucose testing includes a glucose testing measurement module housing, a test strip receptacle formed in the housing, and a connector portion formed in the housing and shaped to permit mechanical removable attachment of the housing to a hand-held computer. Electronics determine the amount of glucose present in a sample of body fluid, when the test strip is positioned in the receptacle and the body fluid is placed on a test strip, and communicate the glucose amount to the hand-held computer via the connector portion.

A U.S. Pat. No. 2,023,0255519 relates to wearable wireless non-invasive blood glucose measurement system. The invention discloses a wearable, wireless, non-invasive blood glucose measurement system comprising an infrared LED-enabled wireless ring interfaced with machine learning software wherein the ring outputs data from the wearer used to determine the blood glucose concentrations of the wearer in real time. The blood glucose data analytics can be subsequently sent to, and displayed on, a smart mobile device, such as an iPhone®, or distributed over a cloud network.

A US patent U.S. Pat. No. 11,185,261B2 relates to the system and method for continuous glucose monitoring (CGM) of blood in a blood vessel of a patient using a non-invasive sensor composed of a patch antenna operating in the Industrial, Scientific and Medical (ISM) Radio band (5.725 GHz-5.875 GHZ). The device determines the blood glucose concentration of the blood in the blood vessel based on the measured shift of the resonant frequency of the non-invasive antenna patch sensor. A radio frequency (RF) synthesizer is used to drive the patch antenna with a fraction of its output coupled to both the antenna and receiver through a directional coupler. In this approach both the transmitted (FWD) and received (REV) power are processed, by demodulating logarithmic amplifiers, which convert the RF signals to corresponding voltages for downstream processing. The resulting voltages are then fed into a microcontroller and the measured shift in resonant frequency is converted to a real-time glucose concentration.

The current invention focuses on a technique that can perform continuous glucose monitoring, which is a significant improvement over existing system. This innovative method will help individuals with diabetes to manage their health more efficiently and comfortably. Moreover, it will allow them to make proactive lifestyle adjustments, which could potentially reduce the risk of diabetes and improve their overall health.

Upon reviewing the existing literature on glucose monitoring systems, it has become clear that there have been several advancements. However, these prior systems have their shortcomings, which make them unsuitable for practical use due to a variety of reasons such as technical difficulties, impracticality, or high costs.

The references/citations relates to our unique patent Alrawi, A., Moghavvemi, M., & Ibrahim, W. (2010). Novel idea to monitor and measure blood hemoglobin noninvasively. AFRICAN JOURNAL OF BIOTECHNOLOGY, 9, 9295-9306.

By using this new technique, individuals with diabetes can monitor their glucose levels more accurately and frequently than was previously possible. This will provide them with real-time information about their glucose levels, which can be used to make timely decisions about their diet, exercise, and medication. The continuous monitoring will also help to identify trends over time, which will allow individuals to make informed decisions about their health.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

The references/citations relates to a patent application Hayashi, Y., Livshits, L., Caduff, A., & Feldman, Y. (2003). Dielectric spectroscopy study of specific glucose influence on human erythrocyte membranes. Journal of Physics D: Applied Physics, 36 (4), 369-374. https://doi.org/10.1088/0022-3727/36/4/307.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of the non-invasive blood glucose monitoring device.

It is also the primary objective of the invention to provide a non-invasive blood glucose monitoring system that utilizes radio frequency (RF) signal interactions with body tissues to infer changes in blood glucose levels. This system operates on the principle that physiological alterations in the body's electrical properties, such as those caused by fluctuating glucose concentrations, can be measured and quantified through RF signal analysis.

It is another objective of the invention to provide a smart system that uses the human body as an RF antenna, wherein the body's inherent electrical characteristics, like intrinsic impedance, are influenced by physiological variations. The device transmits an RF signal at a predefined frequency, typically around 20 MHz, which interacts with body tissues, inducing a measurable response that is indicative of the blood's glucose content.

It is also the objective of the invention to provide a device to measure Voltage Standing Wave Ratio (VSWR) that reflects the mismatch in impedance between the RF signal transmitter and the human body. The body's intrinsic impedance changes due to body cell's plasma layer electric permittivity, which is associated with glucose levels in the blood. The proposed device can accurately assess this mismatch to provide crucial insights into the body's impedance changes and glucose levels. The device can help monitor and manage diabetes and related complications.

It is one of the objectives of this invention is to develop a smart device capable of inducing an RF signal into the body of a user for an adequate time to obtain a stable measurement, thereby collecting data on VSWR and other RF signal characteristics. This real-time data acquisition process allows for immediate analysis and is an essential component in determining blood glucose levels in real-time.

It is a further objective of the invention to provide data processing for glucose level estimation, wherein a plurality of processing steps are involved in transforming the collected RF signal data into an estimate of blood glucose concentration. These steps are executed by a microcontroller or processing unit within the device, which applies algorithms to relate changes in RF signal properties to blood glucose levels.

It is also the objective of the system to provide calibration for personalized accuracy, wherein the calibration process to personalize the device's readings for individual users is provided. This calibration is facilitated by the smartphone application that interfaces with the device which is connected through Bluetooth, guiding users through the initial setup and any necessary adjustments to ensure the system's measurements are accurate and reliable.

Another objective is to provide an assembly that is both convenient and user-friendly, while also incorporating a smartphone application to facilitate visualization and management. Specifically, the smartphone application serves as an interface for users to easily visualize measurement results, manage user profiles, calibration data, and measurement history. The inclusion of such features ultimately enhances the overall usability of the glucose monitoring system.

It is, moreover, the objective of the invention to provide an advanced technique for continuous glucose monitoring that will significantly improve the quality of life for individuals with diabetes, making it easier for them to manage their health and maintain a proactive lifestyle.

Still, another objective is to provide an interactive user interface and operation that simplifies the monitoring system through a smartphone application. The intuitive user interface enables profile selection, device calibration, and real-time glucose level viewing, resulting in a seamless user experience.

Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention and explain various principles and advantages of those embodiments.

FIG. 1.1 illustrates a schematic overview of a non-invasive blood glucose monitoring system, depicting its operational components and their interconnectivity. The system features a patch antenna applied directly to the human skin, operating in concert with an integrated circuit to measure glucose levels through radio frequency signal analysis.

FIG. 1.2 illustrates a visual representation of the hypothesized process by which glucose concentration affects cell membrane permittivity. It outlines the major components involved in the process, including the glucose transporter GluT1, the cellular metabolism pathway leading to ATP production, and the ionic channels that contribute to the membrane's permeability.

FIG. 2.1 illustrates an experimental setup with micro strip patch coupled bio-antennas used for preliminary studies in glucose measurement.

FIG. 2.2 illustrates a waveform comparison between simulated and measured HF pulses, essential for feature extraction in glucose level determination.

FIG. 2.3 illustrates the graph of frequency response in the impedance measurement, showing the impact of different glucose concentrations on impedance values.

FIG. 2.4 illustrates the capacitance of a cell membrane, depicting the cell as a parallel plate capacitor. The diagram explains the relationship between membrane capacitance and the physical characteristics of the cell membrane, including the cross-sectional area and the separation between the plates. The figure includes the formula for capacitance (C) with an example calculation, providing an understanding of the electrical properties of biological cells relevant to non-invasive glucose measurement technologies.

FIG. 2.5 illustrates a schematic representation of the human body modeled as an equivalent circuit of resistors and capacitors. The diagram details how various segments of the body correspond to different electrical components, suggesting how alternating current flows through the body from hand to foot, which is essential for the non-invasive measurement of blood glucose levels by assessing the body's electrical properties.

FIG. 3.1 illustrates the wave behavior of reflection coefficients when interfacing with mediums of differing impedances, a foundational concept in the invention's measurement technique.

FIG. 3.2 illustrates the voltages relevant for calculating the reflection coefficient, pivotal to the device's operational mechanics.

FIG. 3.3 illustrates a basic circuit configuration intended for measuring the reflection coefficient, which is integral to the device's functionality.

FIG. 3.4 and FIG. 3.5 illustrate cross-sectional views of the input system and a body part, respectively, to demonstrate the impact of various biological layers on impedance readings.

FIG. 3.6 illustrates the correlation between the reflection coefficient and relative permittivity, central to the device's glucose measurement capability.

FIG. 3.7 and FIG. 3.8 illustrate the circuit diagram of the oscillator and the output of the oscillator circuit, respectively, essential components of the invention.

FIG. 3.9 and FIG. 3.10 illustrate the amplifier circuit and its output signal, highlighting the necessary amplification of signals for the device's operation.

FIG. 3.11 illustrates the VSWR meter circuit, which is instrumental in assessing the standing wave ratio in the transmission line.

FIG. 3.12 illustrates a utilized within the VSWR meter to sample complex voltages and currents.

FIG. 3.13 illustrates a fasting blood glucose level chart, which categorizes glucose concentrations into various levels and associated risks, from dangerously low to dangerously high levels. The chart provides a visual representation of blood glucose thresholds for conditions such as hypoglycemia, normoglycemia, prediabetes, and diabetes, facilitating the assessment of the user's glycemic state by the non-invasive blood glucose measurement device

FIG. 4.1 and FIG. 4.2 illustrate the working diagram and block diagram of the real-time blood glucose level measurement system, respectively.

FIG. 5.1 illustrates the graphical relationship between Blood Glucose Level (BGL) and Voltage Standing Wave Ratio (VSWR), which is pivotal for the analysis of the device's data.

FIG. 5.2 illustrates the comparative test results between the prototype device and a standard glucometer, affirming the invention's measurement accuracy.

FIG. 5.3 illustrates a wearable prototype of a non-invasive blood glucose monitoring device, designed for unobtrusive and constant measurement. Resembling a wristwatch, this device encapsulates the functionality of the comprehensive monitoring system into a user-friendly form factor, allowing everyday glucose management.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to a non-invasive blood glucose monitoring system that utilizes radio frequency (RF) signals to determine glucose levels in the blood.

In an embodiment of the present invention, the Patch Antenna 425 is meticulously engineered to interface with the Human Skin 455, which maintains characteristic impedance, thus initiating the glucose monitoring process with precision. In this particular embodiment, the Patch Antenna 425 emits radio waves, whose reflection properties change due to blood glucose variations, enabling the VSWR Measurement System 428 to determine the standing wave ratio, a key indicator of glucose levels. The reflection is detected as variations in the Reflected Voltage (Vr) 462. When compared to the Forward Voltage (Vf) 460, this enables the VSWR Measurement System 428 to accurately ascertain the standing wave ratio—an indicator of blood glucose levels. Within the inventive scope of the present invention, the Microcontroller 438 serves as the central processing unit, applying proprietary algorithms to translate intricate digital data into discernible glucose readings. This microcontroller unit connects with an Analog-to-Digital Converters (ADC) 435 and a Digital-to-Analog Converter (DAC) 475, along with a Bluetooth radio subsystem 440.

The Digital-to-Analog Converter (DAC) 475 is utilized to generate a fixed voltage (Vo) 478, which can be adjusted as needed. The Voltage-Controlled Oscillator (VCO) 442 generates radio waves with frequency modulation controlled by the DAC's voltage. Subsequently, the High-Frequency (HF) radio wave is filtered through a High Pass Filter (HPF) 445 to eliminate low-frequency noises and clean up the data. The amplified radio wave, gained by Signal Amplifier 430, is transmitted to the Patch Antenna 425 via the VSWR Measurement System 428. The Patch Antenna 425, a coupling antenna, directly contacts the Human Skin 455. Reflection power is caused by impedance mismatches between the antenna and the human subject's body 455. The VSWR Measurement System 428 outputs two analog signals-Vr 462 and Vf 460 as a result of the reflection, which are sampled by two different ADCs 435.

In another embodiment, wireless communication is enabled via Bluetooth 440, which, along with the Bluetooth Antenna 468, ensures a secure and efficient relay of glucose data to external devices for user access and historical tracking. The external device application is also used to calibrate the system over Bluetooth communications.

Powering such sophisticated operations, the Lipo Battery 470 is governed by the Battery Management System (BMS) & Power Management IC (PMIC) 448, meticulously managing the power supply to ensure sustained functionality of the device.

In a further embodiment of the present invention, the Sweat Probes 458 provides additional data points by measuring skin resistance to counteract wet skin artefacts, thereby enhancing the system's measurement accuracy. The skin resistance is measured using a Wheatstone bridge configuration, with an active Low Pass Filter (LPF) 432 employed to remove noise potentially generated by muscle contractions. The resulting analogue voltage from skin resistance is amplified by Amplifier 465 and sampled by an Analog-to-Digital Converter 435. This embodiment includes Data Handling 452 dedicated to managing calibration and uploading firmware to the Microcontroller 438. Additionally, the USB 450 port is incorporated not only as a charging solution but also as an interface for comprehensive data management, ensuring that the device's software remains up-to-date and operates at peak performance.

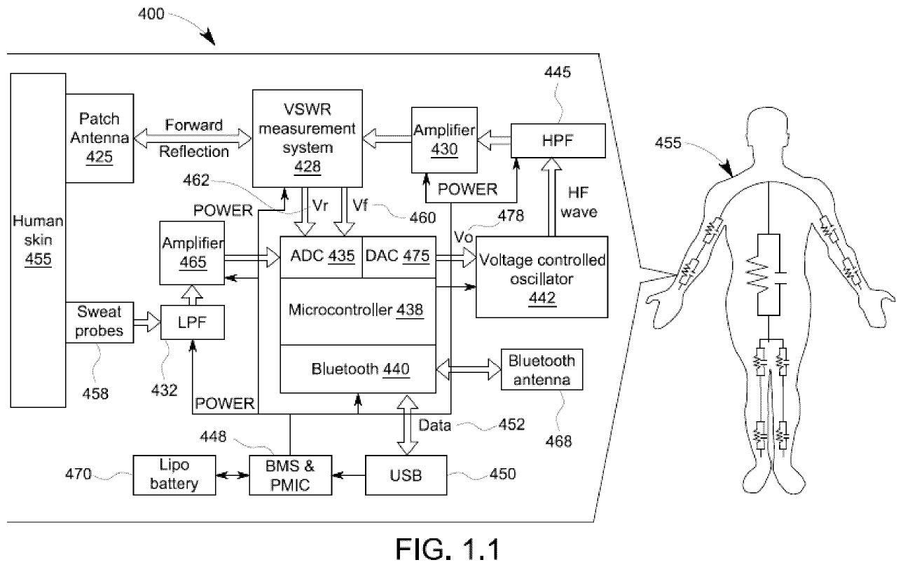

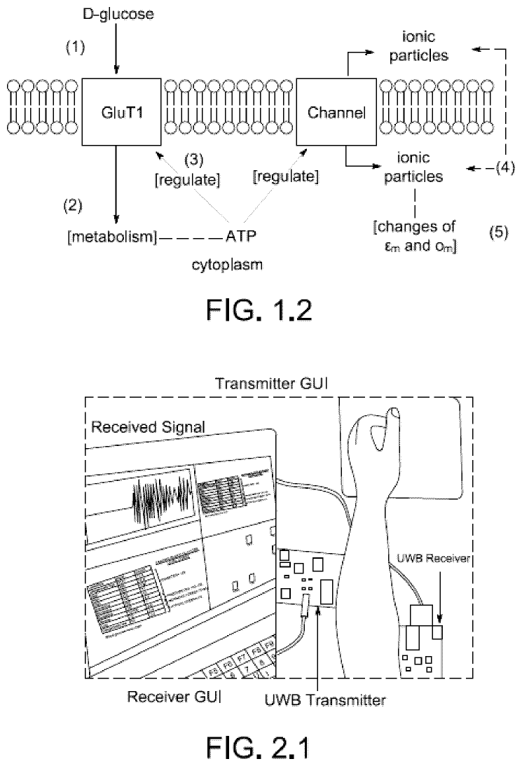

The device can be further understood by the provided Figures and their description. The embodiments herein detail the hypothesized model illustrated in FIG. 1.1, where the D-glucose molecule's interaction with the cell membrane is depicted as a multi-step process that results in changes to the membrane's permittivity. The model begins with D-glucose approaching the cell membrane and interacting with the GluT1 transporter (1). This interaction facilitates the entry of glucose into the cell, where it is metabolized (2), resulting in the production of ATP, a critical energy molecule. The ATP produced then acts as a regulatory agent (3), influencing the activity of various ionic channels within the cell membrane. These channels regulate the flow of ionic particles across the membrane (4), which in turn affects the permittivity of the membrane, specifically the electric permittivity (ε_m) and magnetic permeability (σ_m) (5). The model proposes a direct correlation between the concentration of glucose in the vicinity of the cell membrane and the resulting electrical properties of the membrane, providing a basis for the non-invasive detection of glucose levels as outlined by the invention.

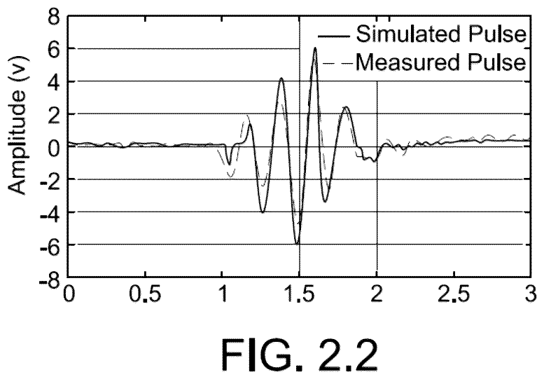

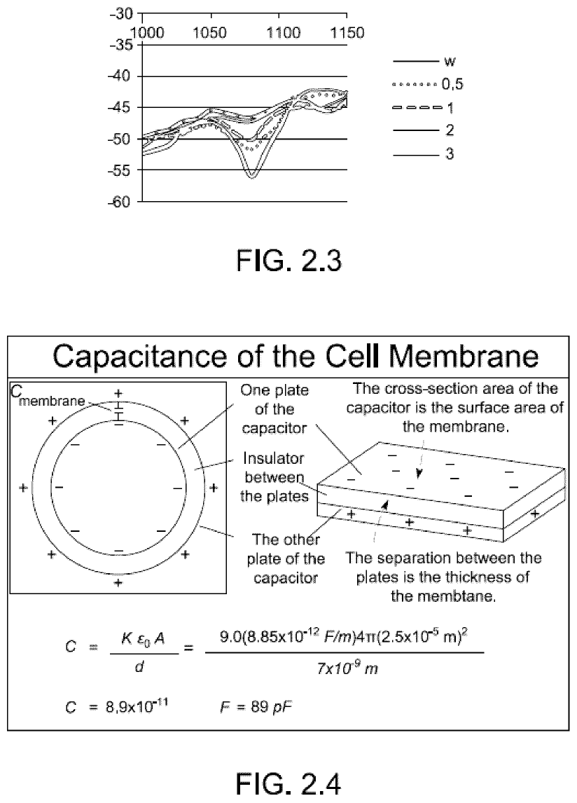

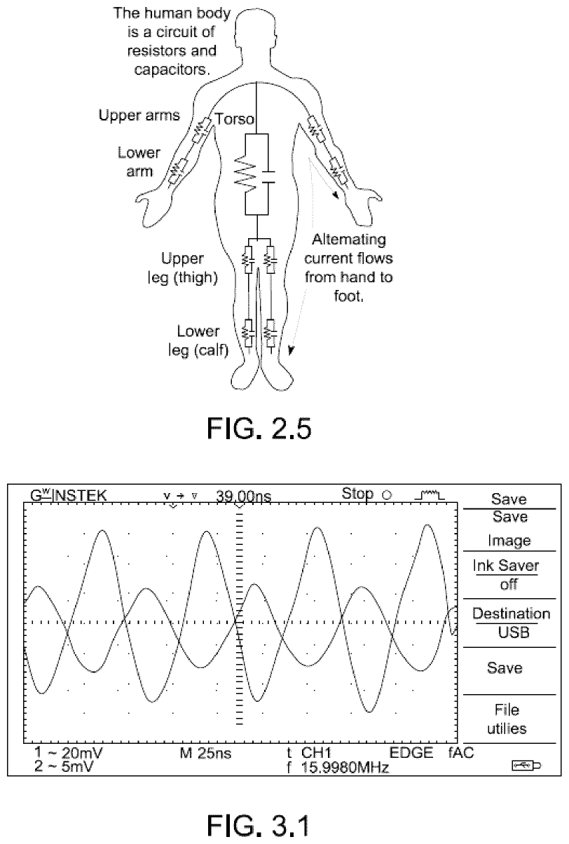

The embodiments herein include FIG. 2.1, which serves as a visual aid to describe an experimental setup using ultra-wideband micro strip patch bio-antennas. This setup is a precursor to the non-invasive glucose measurement technique detailed in this patent, demonstrating the foundational research that underpins the invention's methodology.

In accordance with an embodiment of the present invention, FIG. 2.2 illustrates a critical waveform comparison that is instrumental in the feature extraction process for glucose level determination. The ability to distinguish between simulated and measured UWB pulses is a key aspect of the system's analytical capabilities and forms the basis for the algorithmic processing within the device.

In accordance with another embodiment of the present invention, FIG. 2.4 demonstrates the cell membrane's capacitance modeled as a parallel plate capacitor, which is critical for understanding how electrical properties of the cells' membranes interact with the sensor technology used in the non-invasive glucose measurement device. The figure provides a formula to calculate the membrane's capacitance, which is directly related to the glucose concentration measurements. This understanding allows for refined calibration and enhanced accuracy of the device when measuring glucose levels through changes in the electrical properties of the user's cells.

In an embodiment of the present invention, FIG. 2.5 is employed to illustrate the human body's electrical model as an equivalent circuit, comprising an array of resistors and capacitors. This model is integral to the invention, as it underpins the method by which the device assesses changes in the body's electrical impedance caused by varying glucose concentrations. The equivalent circuit aids in understanding the path and distribution of electrical currents when non-invasively measuring glucose levels, providing insight into the optimization of sensor placement and signal interpretation for accurate glucose level assessment.

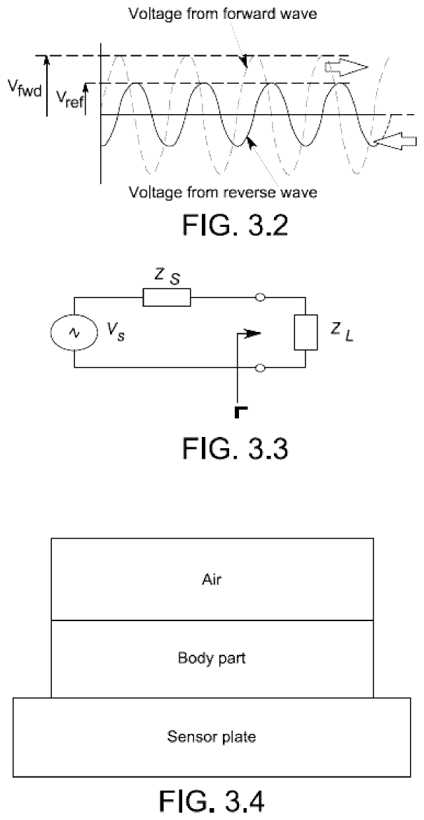

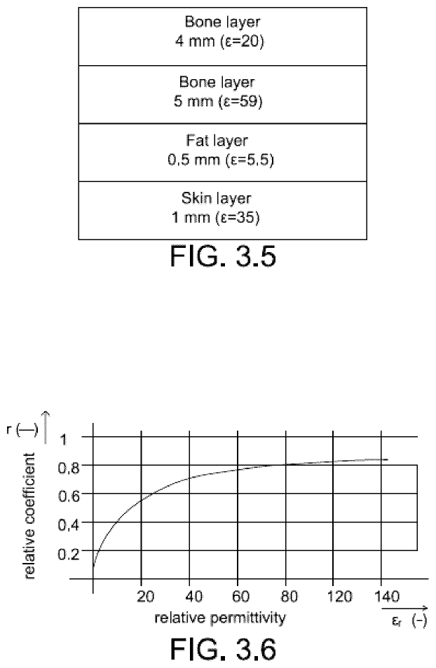

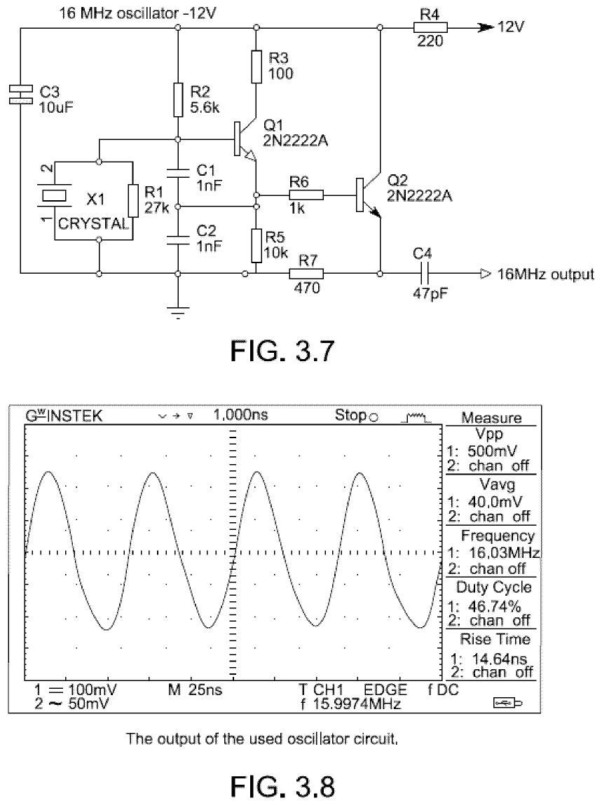

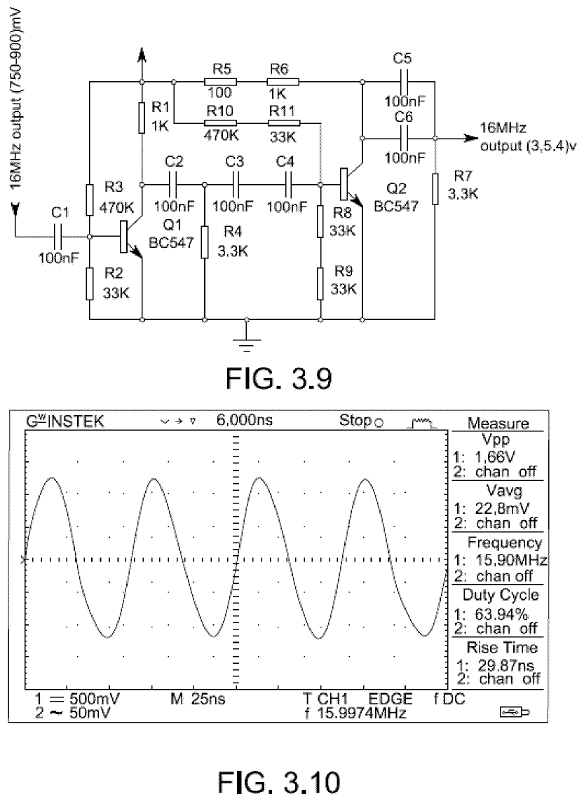

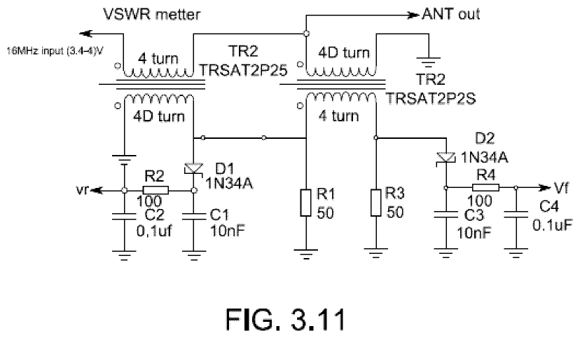

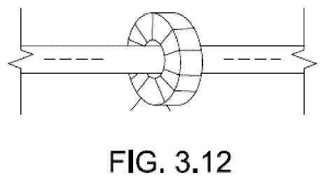

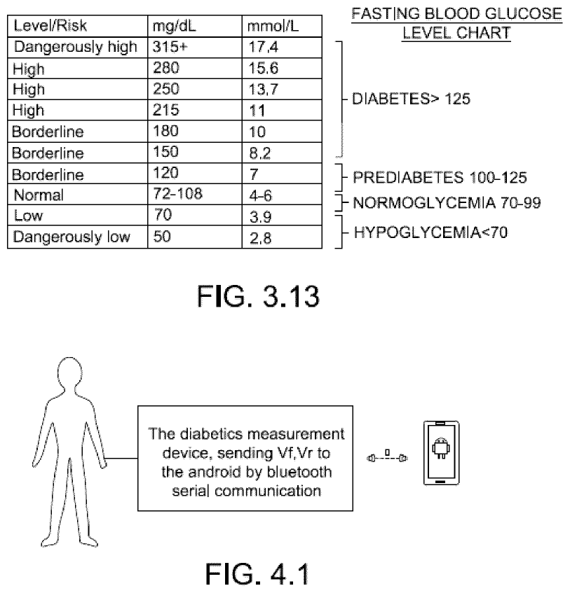

In accordance with an embodiment of the present invention, FIG. 3.1 visually demonstrates the behavior of reflection coefficients as they interact with various impedance mediums. This phenomenon is at the heart of the invention's operational principle, where the body's variable impedance related to glucose concentration is detected and analyzed.

In another embodiment, FIG. 3.2 and FIG. 3.3 together illustrate the voltages and circuit configuration used for calculating the reflection coefficient. The figures detail the intricate electrical interactions within the system, showing how the reflected and incident wave voltages are leveraged to compute the reflection coefficient-a critical parameter in determining glucose levels non-invasively.

The embodiments herein also describe how FIGS. 3.4 and 3.5 illustrate cross-sectional views of the input system and a body part, respectively. These illustrations provide insights into how different biological layers, each with unique impedance properties, interact with the sensor plate. Understanding these interactions allows for the optimization of sensor design and signal processing algorithms to enhance measurement accuracy.

In another embodiment, FIG. 3.6 illustrates the proportional relationship between the reflection coefficient and relative permittivity. The graphical representation in this figure is essential for understanding how changes in the body's permittivity, due to varying glucose levels, influence the reflection coefficient. This understanding is pivotal for the accurate conversion of electrical signals into glucose level readings.

The embodiments herein include FIGS. 3.8 and 3.9, which illustrate the detailed circuitry of the oscillator and the resulting output waveform, respectively. These components are critical for generating the high-frequency signals required by the system, with the oscillator's stability and output power directly influencing the precision of glucose measurements.

In accordance with an embodiment of the present invention, FIGS. 3.10 and 3.11 illustrate the amplifier circuit and its output signal. The amplifier's role in boosting the signal strength is crucial for ensuring that the sensor plate receives adequate signal power for accurate impedance matching and subsequent reflection coefficient calculation.

In another embodiment, FIG. 3.11 is dedicated to illustrating the VSWR meter circuit, an essential component for quantifying the standing wave ratio in the transmission line. This figure details the internal configuration of the VSWR meter, which is central to evaluating the effectiveness of impedance matching between the transmission antenna and the human body.

The embodiments herein describe the various methods and components used within the VSWR meter to sample complex voltages and currents, as shown in FIGS. 3.13 through 3.15. These figures elucidate the sophisticated means by which the system isolates forward and reverse wave components, a critical step in the determination of VSWR and, by extension, the glucose concentration.

In accordance with an embodiment of the present invention, FIG. 3.16 illustrates a fasting blood glucose level chart, categorizing glucose concentrations into various levels and risks associated with them. This chart is utilized by the device's algorithm to accurately classify the glucose measurements obtained, providing a clear and immediate understanding of the user's glycemic state. A plurality of glucose concentration ranges is shown, from dangerously low to dangerously high, which allows the device to alert users according to the urgency of their situation.

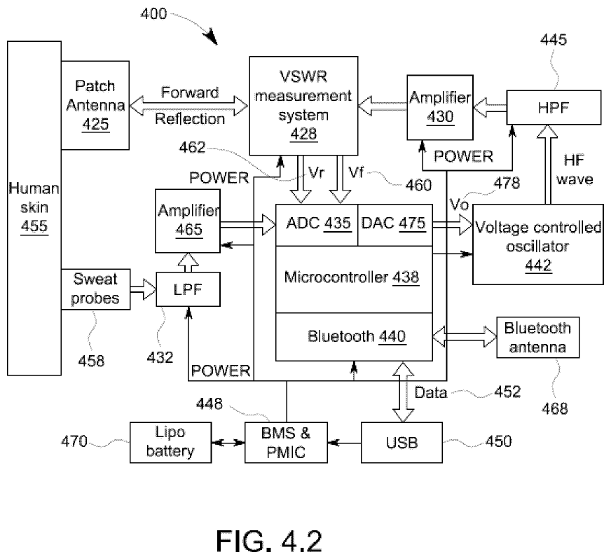

In another embodiment, FIGS. 4.1 and 4.2 together provide a comprehensive illustration of the working principle and block diagram of the real-time blood glucose level measurement system. These figures present an overview of the system's architecture, elucidating the pathway from physical sensing to data processing and final output display.

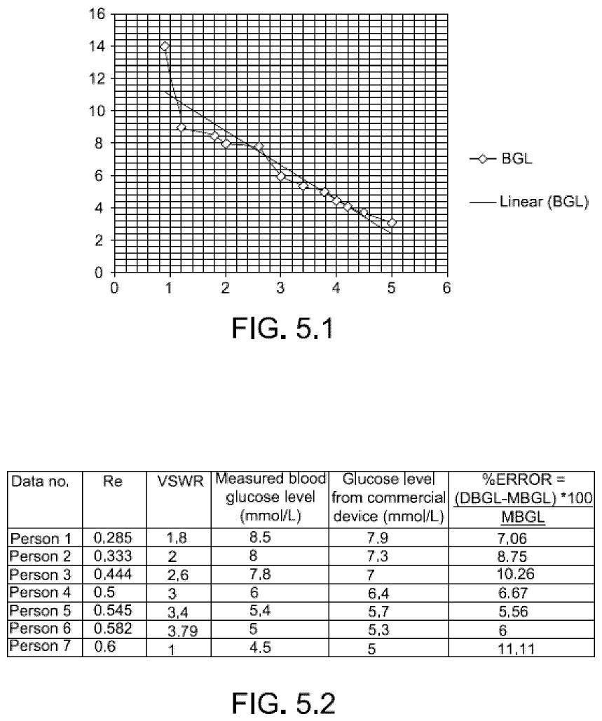

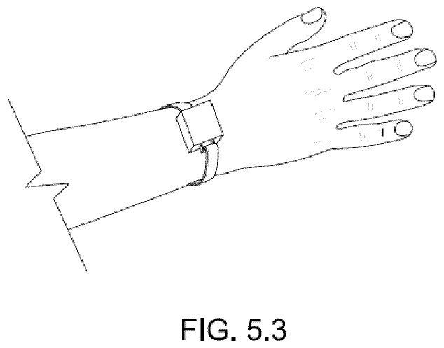

The embodiments herein utilize FIG. 5.1 to illustrate the empirically derived correlation between Blood Glucose Level (BGL) and Voltage Standing Wave Ratio (VSWR). This relationship forms the basis for the device's interpretive algorithms, which convert VSWR readings into meaningful glucose level data.

In accordance with an embodiment of the present invention, Table 5.1 illustrates the results of comparative testing between the prototype device and a commercial glucometer. The table is instrumental in validating the invention's measurement accuracy, providing a quantitative basis for the system's performance evaluation.

In another embodiment, FIG. 2.1 illustrates an experimental setup showcasing the ultra-wideband (UWB) technology operated in the validation of the non-invasive blood glucose monitoring device. The image illustrates a UWB transmitter attached to a human arm, which is part of the system used to collect data on reflected signals that are crucial in determining glucose levels. The accompanying graphical user interfaces (GUI) display the transmitted and received signal profiles, underscoring the device's capability to capture detailed physiological reactions for glucose monitoring.

A plurality of innovations is presented in the oscillator and amplifier design, crucial for the device's functionality. The oscillator, capable of producing a 16 MHz frequency signal as required for the operation of the device, is detailed in FIG. 3.7. The subsequent amplifier design, as shown in FIG. 3.9, illustrates how the signal strength is increased to ensure effective measurement, highlighting the integral role of these components in the system's architecture.

The detailed description of the invention further includes the VSWR meter's functionality, which is instrumental in measuring the standing wave ratio in a transmission line, providing insights into the degree of mismatch between the line and its load. This feature is depicted in FIG. 3.11, where the meter's ability to sense forward and reverse waves through directional couplers is elucidated.

The comprehensive system architecture and prototype testing, shown in FIG. 4.2, present an overview of the entire system from the sensor plate's interaction with the human body to the SWR measurement system's processing of data. The Bluetooth module's role in transmitting information to a display output or for further data collection and processing underlines the seamless and integrated nature of the device's design.

In another embodiment, the interpretation of measurement data is provided, with FIG. 5.1 visually representing the inverse relationship between BGL and VSWR. This graphical representation forms the basis for the device's algorithm to calculate glucose levels, reflecting the system's ability to transform raw data into meaningful health indicators.

The embodiments herein also include a comparative analysis between the prototype device and a commercial glucometer, as illustrated in Table 5.1 and FIG. 5.1. This analysis is essential for establishing the non-invasive device's relative accuracy and validating its performance, thereby substantiating the invention's potential to revolutionize glucose monitoring practices.

A plurality of operational workflows for the device are illustrated in FIG. 4.1, summarizing the diabetic measurement process. This workflow not only demonstrates the critical hardware components and their interconnections but also the user-friendly aspect of the device, showing how it functions in practice to measure and transmit blood glucose level data.

In another embodiment, the present invention contemplates the precision of the algorithm used for glucose estimation. The algorithm's design, which incorporates quadratic ration interpolation, factors in the complex relationship between BGL and VSWR to deliver highly accurate glucose readings, as can be anticipated from the data plotted in FIG. 5.1.

The embodiments herein further provide a detailed examination of the engineering of the device's electrical components. The circuit diagrams and layouts, such as those detailed in FIG. 3.7 and FIG. 3.9, reveal the careful consideration given to the design and function of each component, ensuring the overall reliability and efficiency of the device.

In accordance with an embodiment of the present invention, a comprehensive system calibration approach is outlined. This process includes a meticulous step-by-step method for calibrating the device to match individual physiological characteristics, which is essential for the personalization and precision of the non-invasive glucose measurements.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A non-invasive glucometer system comprising:
a high-frequency radio signal generator for emitting a signal towards a subject's body;
a stripe transmission antenna coupled to the high-frequency radio signal generator for directing the emitted signal;
a sensor configured to detect reflected signals from the subject's body and determine a Voltage Standing Wave Ratio (VSWR) indicative of impedance mismatches; and
an analyzer system configured to correlate the VSWR with the subject's blood glucose level.
The system of claim 1, wherein the analyzer system further comprises a calibration mechanism for personalizing the system to the subject's body impedance characteristics upon initial use.
The system of claim 1 or 2, wherein the high-frequency radio signal generator operates at a frequency optimized for penetration of human tissue without causing harm.
The system of any preceding claims, wherein the stripe transmission antenna is designed to minimize inductive and capacitive reactance to enhance signal transmission efficiency.

2. A method for non-invasive measurement of blood glucose levels in a subject, the method comprising the steps of:
emitting a high-frequency radio signal towards the subject's body;
capturing a reflected signal from the subject's body;
calculating a VSWR based on the emitted signal and the reflected signal; and
determining the blood glucose level of the subject from the VSWR.
The method of claim further comprising a calibration step involving adjusting the system based on the subject's baseline body impedance characteristics.

3. The method of claim, wherein the high-frequency radio signal is generated at a frequency that allows for accurate detection of changes in the subject's body impedance without adversely affecting biological tissue.
The method of any of claims, wherein the determination of the blood glucose level is further refined based on a plurality of measurements taken over a set time period to account for transient impedance fluctuations.
A non-invasive glucometer system for determining blood glucose levels, the system operatively configured to:
utilize a microcontroller for processing signals related to the subject's body radio wave impedance;
implement Bluetooth communication for transmitting data to a secondary device for further analysis; and
present measurement results on the secondary device through a dedicated application interface.
The system of claim, wherein the microcontroller is programmed to employ quad ration interpolation based on previous measurements to enhance the accuracy of blood glucose level determination.
The system of any preceding claims, wherein the glucometer system includes a user interface application on a secondary device, the application configured to:
receive and display real-time measurement data;
guide the subject through the calibration process; and
store historical data for ongoing monitoring and analysis.
The system of any preceding claims, wherein the glucometer system is configured to perform measurements and provide results in a format compatible with common diabetes management protocols.
